(19) United States
(12) Patent Application Publication
(10) Pub. No.: US 2026/0096827 A1
(43) Pub. Date: Apr. 9, 2026

(54) CAPTURE DEVICES AND RELATED SYSTEMS AND METHODS OF USE

(71) Applicant: Boston Scientific Scimed, Inc., Maple Grove, MN (US)

(72) Inventors: Majd SLEEM, Natzrat Ilit (IL); John SUBASIC, Sutton, MA (US)

(73) Assignee: Boston Scientific Scimed, Inc., Maple Grove, MN (US)

(21) Appl. No.: 19/351,652

(22) Filed: Oct. 7, 2025

Related U.S. Application Data
(60) Provisional application No. 63/704,690, filed on Oct. 8, 2024.

(51) Int. Cl. A61B 17/221; A61B 17/00; A61B 17/22

(52) U.S. Cl. CPC A61B 17/221; A61B 2017/00367; A61B 2017/00743; A61B 2017/00867; A61B 2017/22074; A61B 2017/2212

(57) ABSTRACT

Capture devices and related systems are described, including a medical device that includes a handle coupled to an end effector. The end effector may be formed from a first wire and a second wire each forming a coil. The coil of the first wire may be proximal to the coil of the second wire. The first wire and the second wire each include a shape memory material, such that the end effector includes a collapsed configuration when constrained within a sheath and an expanded configuration when unconstrained by the sheath. While in the expanded configuration, the first wire and the second wire may be movable relative to each other top provide a space, e.g., an opening, between the coil of the first wire and the coil of the second wire.

### CROSS-REFERENCE TO RELATED APPLICATIONS

This application claims the benefit of priority to U.S. Provisional Application No. 63/704,690, filed on October 8, 2024, which is incorporated by reference herein in its entirety.

### TECHNICAL FIELD

Various aspects of this disclosure relate generally to medical devices, systems, and procedures. Particular aspects relate to medical devices and systems for removing material, e.g., obstructions, from one or more body lumens and/or passageways, among other aspects.

### BACKGROUND

During noninvasive procedures, a physician may use a capture device (e.g., a basket or grasper) to engage objects in a body. For example, in some urology procedures, the physician may advance a sheath into a kidney, extend a basket from the sheath, engage a stone in the kidney with the basket, and retract the basket to capture the stone. Often, a capture device may be deployed through a working channel of an endoscope or other insertable device. For example, a ureteroscope may be placed in the ureter or kidney, and a capture device may be deployed through a working channel of the ureteroscope and used to “grab” a stone or tissue for removal. A plurality of stones and/or stone fragments may need to be removed during the procedure, requiring the basket to be extended numerous times.

### SUMMARY

Each of the aspects disclosed herein may include one or more features described in connection with any of the other disclosed aspect.

The present disclosure includes medical devices, e.g., capture devices, and related systems and methods of use. For example, the present disclosure includes a medical device comprising a handle coupled to an end effector, wherein the end effector is formed from a first wire and a second wire. Each of the first wire and the second wire may form a coil, optionally wherein the coil of the first wire is proximal to the coil of the second wire. The first wire and the second wire each may comprise a shape memory material such as nitinol, such that the end effector includes a collapsed configuration when constrained within a sheath and an expanded configuration when unconstrained by the sheath. The first wire and the second wire may be movable relative to each other to provide a space, e.g., an opening, between the coil of the first wire and the coil of the second wire while in the expanded configuration.

According to some aspects of the present disclosure, the handle may include a first actuator operably coupled to the first wire. The first actuator may be configured to move the first wire relative to the second wire. The handle may include a second actuator operably coupled to the second wire. The second actuator may be configured to move the second wire relative to the first wire. The first wire and the second wire may be movable independent of each other, e.g., via the first actuator and the second actuator, respectively. When the end effector is in the expanded configuration, the first wire and the second wire may be spaced apart at a proximal end of the end effector to permit insertion of a treatment fiber such as a laser fiber into the end effector. In some aspects, a pitch of the coil of the first wire and/or of the coil of the second wire may range from about 1.5 mm to about 2 mm. In the expanded configuration, the end effector may include an ellipsoidal shape. Further, for example, in the expanded configuration, a portion of the second wire may extend through an interior volume of the coil of the first wire. In the expanded configuration, the end effector may define a volume configured to receive a target material. The space between the coil of the first wire and the coil of the second wire may provide an opening of the end effector, e.g., into the volume. A size of the opening may be variable by moving the first wire relative to the second wire.

In some examples, the medical device includes the sheath. For example, the sheath may define a lumen that receives the first wire and the second wire. The handle may include a port defining a channel in communication with the lumen of the sheath.

The present disclosure also includes a medical device comprising a handle, an end effector, and a sheath coupled to the handle, wherein the handle includes a first actuator and a second actuator. The end effector may be formed from a first wire and a second wire, each comprising a shape memory material forming a coil. The coil of the first wire may be proximal to the coil of the second wire. The first wire may be operably coupled to the first actuator and the second wire may be operably coupled to the second actuator. The end effector may be slidable along a lumen of the sheath. The end effector may include a collapsed configuration when constrained within the sheath and an expanded configuration when unconstrained by the sheath. The first wire and the second wire may be movable relative to each other via the first actuator and the second actuator, respectively, to provide a space such as an opening between the coil of the first wire and the coil of the second wire while in the expanded configuration.

According to some aspects of the disclosure, the handle may include a port defining a channel in communication with the lumen of the sheath. In the expanded configuration, the end effector may define a volume configured to receive a target material. The volume may include an opening defined by a distance between the coil of the first wire and the coil of the second wire. A size of the opening may be variable by moving the first wire relative to the second wire. The coil of the second wire may be distal to the coil of the first wire. Each of the first actuator and the second actuator may include a slider, e.g., for moving the coil of the first wire and the coil of the second wire.

The present disclosure also includes a method of treating a subject, e.g., the method comprising navigating a distal end of a medical device through a urinary tract of the subject to a treatment site, and extending an end effector of the medical device through a lumen of a sheath of the medical device to exit the lumen, thereby transitioning the end effector from a collapsed configuration to an expanded configuration. As described above and elsewhere herein, the end effector may include a first wire and a second wire each comprising a shape memory material such as nitinol. Each of the first wire and the second wire may form a coil (as a predetermined shape) in the expanded configuration. The coil of the first wire may be proximal to the coil of the second wire. The method may include moving one of the first wire or the second wire away from the other of the first wire or the second wire, and capturing a target material within a volume of the end effector via an opening between the coil of the first wire and the coil of the second wire. The method may comprise moving one of the first wire or the second wire towards the other of the first wire or the second wire to close the opening, e.g., for removing the target material from the subject.

As mentioned above and elsewhere herein, the medical device may comprise a handle coupled to a sheath, and a first actuator operably coupled to the first wire and a second actuator operably coupled to the second wire. The first wire may be movable relative to the sheath and independent of movement of the second wire. The method may further comprise inserting a treatment fiber into the lumen of the sheath, and extending the treatment fiber proximate the end effector before or after capturing the target material within the volume of the end effector.

### BRIEF DESCRIPTION OF THE FIGURES

The accompanying drawings, which are incorporated in and constitute a part of this application, illustrate exemplary aspects that, together with the written description, explain the principles of this disclosure. The figures depict exemplary aspects according to this disclosure, as follows:

FIG. 1 depicts an exemplary medical device with an end effector in a collapsed configuration, according to aspects of this disclosure

FIG. 2 depicts the medical device of FIG. 1 in an expanded configuration of the end effector, according to aspects of this disclosure.

FIG. 3 depicts the end effector of the medical device of FIG. 1 in the expanded configuration and in position for capturing a material, according to aspects of this disclosure.

### DETAILED DESCRIPTION

Particular aspects of the present disclosure are described in greater detail below. The terms and definitions provided herein control, if in conflict with terms and/or definitions incorporated by reference. The terms “proximal” and “distal” are used herein to refer to the relative positions of the components of exemplary medical devices. As used herein, “proximal” refers to a position relatively closer to the exterior of the body or closer to an operator (e.g., physician or other medical professional) using the medical device (see proximal “P” and distal “D” directional arrows in the figures). In contrast, “distal” refers to a position relatively further away from the operator using the medical device, or closer to the interior of the body.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Although scopes and ureteroscopes are referenced herein for illustration purposes, it will be appreciated that the disclosure encompasses any suitable medical device configured to allow an operator to access and view internal body anatomy of a subject and/or to deliver medical instruments, such as, for example, biopsy forceps, graspers, baskets, snares, probes, scissors, capture/retrieval devices, lasers, and other tools, into the subject’s body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device may be used in connection with the features described herein.

Aspects of various medical devices that include end effectors useful for capturing a material within the body are now described. The end effector may comprise a shape memory material that permits the end effector to transition between a collapsed configuration and an expanded configuration. For example, in the expanded configuration, the end effector may adopt a predetermined shape, e.g., a coiled shape. The end effector may comprise two wires, each having a distal portion that has a coiled predetermined shape. The wires may be movable independent of each other to allow a material to be captured within the end effector, e.g., between two coils.

Some aspects are described with reference to medical procedures, wherein a capture device is extended from a sheath within the body to engage an object at the treatment site. In urology procedures, for example, a medical device may be inserted into the urethra, moved through the bladder and ureter, and advanced into a calyx of a kidney; and an end effector of the medical device may be extended distally from a sheath and/or a working channel to engage one or more stones and/or stone fragments located in the calyx. References to a particular type of procedure, such as a urology procedure; capture device, such as a basket or grasper; organ, such as a kidney; and/or object, such as a stone or stone fragment, are provided for convenience and not intended to limit this disclosure. Accordingly, the concepts described herein may be utilized for any analogous medical device or system.

Reference will now be made in detail to examples to help illustrate aspects of the present disclosure through the accompanying drawings. Wherever possible, the same reference numbers will be used throughout the drawings to refer to the same or like parts.

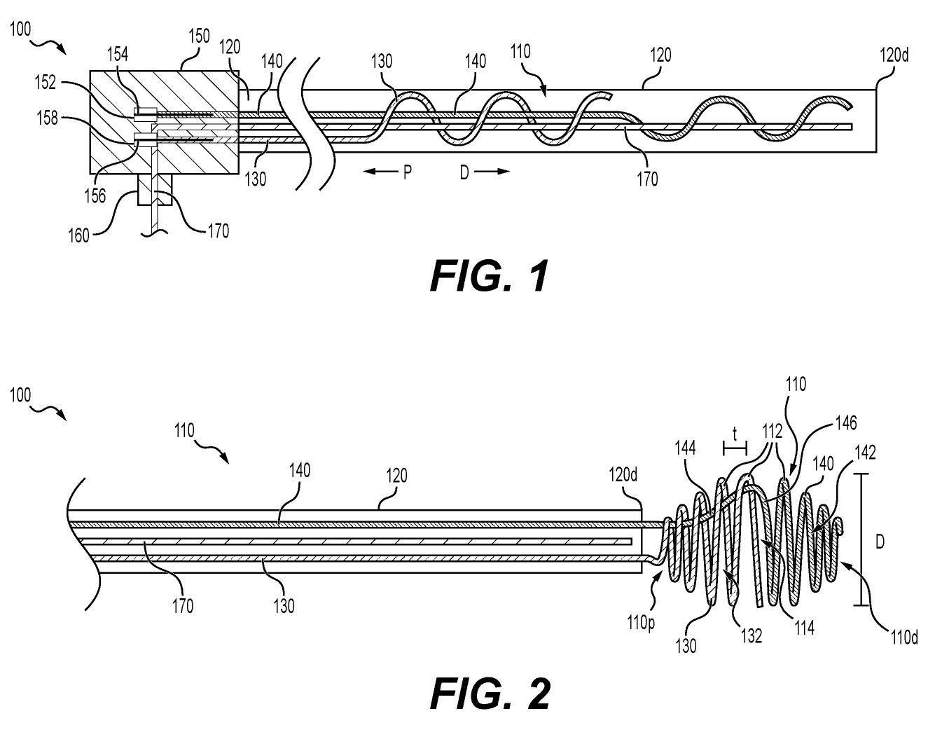

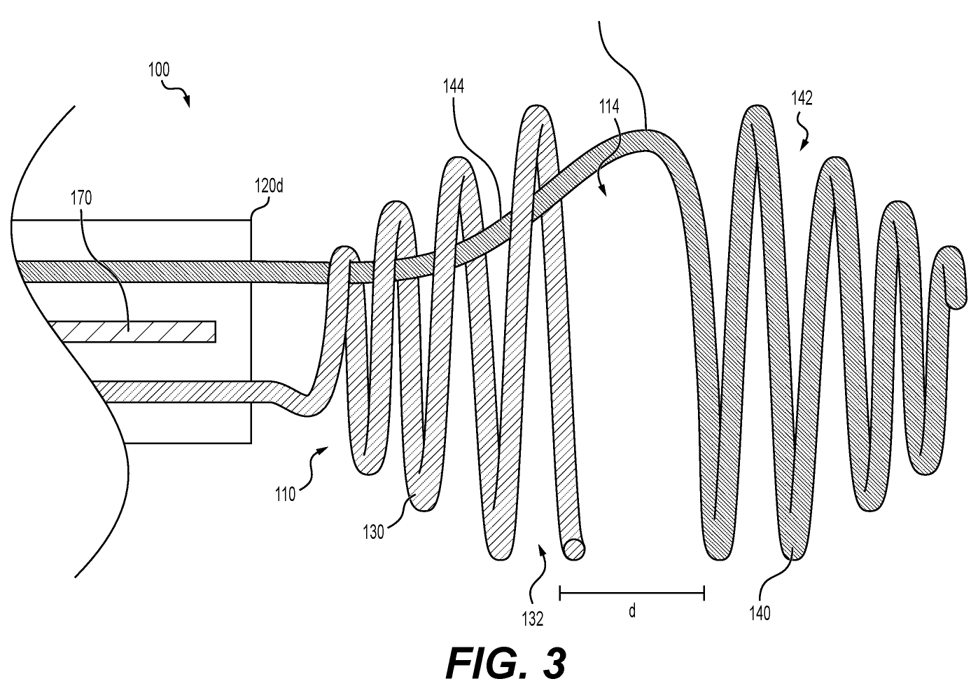

FIG. 1 depicts an exemplary medical device 100 that includes a handle 150 at a proximal end of medical device 100 and a sheath 120 coupled to and extending in a distal direction from a distal end of handle 150. A distal end of medical device 100 includes an end effector 110. End effector 110 comprises a first wire 130 and a second wire 140 each comprising a shape memory material and having distal portions formed into a predetermined shape. For example, each of first wire 130 and second wire 140 may be coiled (see FIGS. 2 and 3) but be capable of lengthening or uncoiling when constrained within walls of a tube or a sheath (see FIG. 1). For example, at least a portion of first wire 130 and second wire 140 are disposed within a lumen of sheath 120. First and second wires 130, 140 may be movable, e.g., slidable, along the lumen of sheath 120 and may be movable, e.g., slidable relative to each other. For example, first and second wires 130, 140 may be independently slidable relative to each other.

Referring to FIG. 1, handle 150 may be configured to selectively extend distally and retract proximally one or both of first and second wires 130, 140. For example, handle 150 may include one or more actuators operably coupled to end effector 110, for sliding first and second wires 130, 140 in a proximal direction and the distal direction. The one or more actuators may be independently moveable / slidable relative to each other. In the example illustrated, the handle 150 includes a first actuator 152 and a second actuator 158. First actuator 152 may be operably coupled to first wire 130 such that urging first actuator 152 distally may slide first wire 130 distally and urging first actuator 152 proximally may slide first wire 130 proximally. Similarly, second actuator 158 may be operably coupled to second wire 140 such that urging second actuator 158 distally may slide second wire 140 distally and urging second actuator 158 proximally may slide second wire 140 proximally.

Each actuator 152, 158 may include a suitable user element such as a slider, knob, switch, lever, etc., for the operator to trip to control movement of end effector 110. According to some aspects of the present disclosure, first actuator 152 and second actuator 158 may each include a slider. Handle 150 may include one or more channels or slots defined through a housing of handle 150 from which actuators 152, 158 extend and are moveable longitudinally along the length of the respective channel or slot. For example, first actuator 152 may extend through a first slot 154 and second actuator 158 may extend through a second slot 156, each slider being movable proximally and distally to control corresponding movement of first and second wires 130, 140.

Handle 150 may further include a port 160 in communication with the lumen of sheath 120. While port 160 as shown in FIG. 1 is a side port, this is exemplary only. For example, in other examples, port 160 may extend from a proximal end of handle 150. Port 160 may be configured to receive a medical instrument such as a treatment fiber 170 to be extended through sheath 120. According to some aspects of the present disclosure, medical device 100 may include one or more other lumens or channels in communication with a corresponding port or ports of handle 150. Such additional lumen(s) or channel(s) and port(s) may be fluidly coupled to a vacuum source or fluid source, such that medical device 100 may be used to provide fluid (e.g., irrigation fluid such as saline, sterile water, etc.) or deliver suction to a treatment site. For example, sheath 120 may correspond to a working channel of a scope or other medical device that includes one or more other working channels available for fluid or suction.

During an exemplary procedure, treatment fiber 170 may be extended through the lumen of sheath 120 to extend distally beyond a distal end 120d of sheath 120 and may be retracted proximally such that treatment fiber 170 is proximal distal end 120d. In some instances, treatment fiber 170 may be extended into end effector 110. Treatment fiber 170 may include a laser fiber for delivering laser energy, an optical fiber for delivering electromagnetic radiation (e.g., visible light, radio waves, UV light, etc.), a viewing fiber with an imager or camera for viewing calculi or a target site, an ultrasonic fiber for delivering ultrasonic energy, and/or an electrode for electrocautery, for example. For example, during an exemplary procedure, treatment fiber 170 may include a laser fiber for delivering laser energy to a target (e.g., a kidney stone or other materials found within a body of a subject) captured within end effector 110.

As mentioned above and shown in FIGS. 1-3, end effector 110 is formed from distal portions of first wire 130 and second wire 140 having predetermined shapes. A distal portion of first wire 130 may form a first coil 132 (also referred to herein as a coil 132 of first wire 130) and a distal portion of second wire 140 may form a second coil 142 (also referred to herein as a coil 142 of second wire 140). A portion of first and second wires 130, 140 proximal to respective coils 132, 142 may have different predetermined shapes; for example, proximal portions of first and second wires 130, 140 may be substantially straight (e.g., not coiled) and/or generally parallel with a longitudinal axis of medical device 100.

As shown in FIG. 2, each of coils 132, 142 may include a plurality of windings 112 with a pitch, t, between of adjacent windings 112 and a maximum diameter, D, relative to the central longitudinal axis. The pitch, t, between adjacent windings 112 may range from about 1.5 mm to about 2 mm. According to some aspects, the pitch, t, between adjacent windings 112 may be sized to prevent relatively larger stone fragments created by lasing a target material (e.g., a kidney stone) captured within end effector 110 from exiting end effector 110 and/or to allow smaller stone fragments (e.g., dust particles) to exit end effector 110. In other words, the pitch, t, may be sufficiently sized to allow a predetermined maximum size of stone or stone fragment to exit end effector 110 between windings 112 and to prevent a predetermined minimum size of stone or stone fragment from exiting end effector 110. In some aspects, the maximum diameter, D, may range from about 5 mm to about 15 mm, such as about 10 mm. First wire 130 and second wire 140 may be configured such that first coil 132 is proximal to second coil 142.

As mentioned above, first and second wires 130, 140 each comprise a shape memory material capable of returning to a predetermined shape. Thus, for example, end effector 110, and each of first and second wires 130, 140 individually, include a collapsed configuration (FIG. 1) and an expanded configuration (FIGS. 2-3). The shape memory material may include nitinol (nickel titanium alloy), other shape memory alloys, and/or any other shape memory materials, and combinations thereof. In the expanded configuration and the collapsed configuration, first and second wires 130, 140 may be spaced radially apart at a proximal end of end effector 110 and/or along a length of sheath 120 to permit insertion of treatment fiber 170 between first and second wires 130, 140 and into end effector 110.

In FIG. 1, end effector 110 is shown constrained within sheath 120 in the collapsed configuration. In the collapsed configuration, end effector 110 (e.g., first and second wires 130, 140) is proximal to distal end 120d of sheath 120 and prevented from expanding, via its shape memory properties, to the predetermined shape of the expanded configuration shown in FIGS. 2 and 3. In the collapsed configuration, compared to the expanded configuration, the pitch, t, of coils 132, 142 may be increased while the number of windings 112 and maximum diameter, D, is decreased.

When end effector 110 is moved distally beyond distal end 120d, end effector is unconstrained by sheath 120 and may automatically assume its predetermined shape in the expanded configuration. The predetermined shape(s) of first and second wires 130, 140 and of end effector 110 may be ellipsoidal in shape or any other suitable shape such as spherical, a conical, or egg-shaped. First coil 132 may extend distally from a proximal end 110p of end effector 110 to the distal end of first coil 132. In its shape memory/predetermined shape, first coil 132 may include a variable diameter increasing in the proximal to distal direction. For example, the diameter of first coil 132 may increase from a minimum diameter at proximal end 110p of end effector 110 to maximum diameter D at or proximate the distal end of first coil 132. Second coil 142 may similarly include a variable diameter decreasing in diameter in the proximal to distal direction. For example, the diameter of second coil 142 may decrease from maximum diameter D at or proximate a proximal end of second coil 142 to a minimum diameter at distal end 110d of end effector 110.

In the expanded configuration, end effector 110 may define an inner volume 114. As shown in FIG. 3, volume 114 may be defined by first coil 132 and second coil 142. Volume 114 may be sufficient to contain materials such as kidney stones and fragments thereof. Second wire 140 may include an angled/curved portion 144 and a bend portion 146. Curved portion 144 may be proximal to second coil 142 and bend portion 146 may be disposed between curved portion 144 and second coil 142. Relative to a central longitudinal axis of medical device 100, curved portion 144 may extend radially outward. A portion of second wire 140 may extend through a portion of volume 114 defined by first coil 132 (e.g., an interior volume of first coil 132). For example, curved portion 144 may be radially inward of coil 132 and may extend adjacent to radially-inward facing surfaces of windings 112 of coil 132. Bend portion 146 may be positioned at or distal to the distal end of coil 132. Bend portion 146 may include an angle so that a portion of second wire 140 distal to bend portion 146 is generally perpendicular to the central longitudinal axis.

While end effector 110 is in the expanded configuration during a medical procedure, treatment fiber 170 may be extended distally beyond distal end 120d of sheath 120 and into volume 114. For example, according to aspects where treatment fiber 170 includes a laser fiber, treatment fiber 170 may be extended distally through a portion of volume 114 or be extended to be proximate volume 114 such that treatment fiber 170 is positioned to deliver laser energy to a material target within volume 114.

End effector 110 may be transitioned between the collapsed configuration (FIG. 1) and the expanded configuration (FIG. 2) via actuators 152, 158 as discussed above. The shape-memory properties of first and second wires 130, 140 may allow the end effector 110 to transition between the collapsed configuration and the expanded configuration multiple times during one or more medical procedures. The ability to transition end effector 110 between configurations multiple times during one or more operations may extend the useful life of medical device 100, e.g., providing the ability to capture various materials and fragments during the same procedure.

From the collapsed configuration shown in FIG. 1, end effector 110 may transition to the expanded configuration shown in FIGS. 2 and 3 as coils 132, 142 expand, increasing the number of windings 112 and decreasing the pitch, t, between adjacent windings 112. When end effector 110 is retracted proximally relative to sheath 120 to the collapsed configuration, coils 132, 142 may contact an opening of sheath 120 at distal end 120d, contract to fit within the lumen of sheath 120, decreasing the number of windings 112 and increasing the pitch, t, between adjacent windings 112.

As mentioned above, first and second wires 130, 140 may be movable relative to each other. Thus may assist in capturing a material within volume 114, e.g., by increasing the distance between first and second wires 130, 140. End effector 110 may include a distance, d, (e.g., a space or opening) between the proximal end of second coil 142 and the distal end of first coil 132. When first and second coils 132, 142 are close together, volume 114 of end effector 110 may be substantially contained to entrap material therein. Accordingly, the distance, d, between coils 132, 142 may be at a minimum distance in this configuration. For example, the distance, d, may be less than or equal to the pitch, t, of windings 112 in the expanded configuration. First and second wires 130, 140 may be moved away from each other via actuators 152, 158 to increase the distance, d, such that a material may more easily enter volume 114. For example, the distance, d, may be greater than the pitch, t, of windings 112 and/or greater than a dimension of the material to be captured.

The user may release the captured material from volume 114 by moving first and second wires 130, 140 away from each other. While the material is captured within volume 114, the user may extend treatment fiber 170 distally to a location proximate end effector, optionally within volume 114. The user may actuate treatment fiber 170 to deliver laser energy to the captured material entrapped within volume 114. In the case of a laser fiber, for example, the laser fiber may fragment the material to more easily transport the fragments proximally for removal from the patient.

In an exemplary medical procedure utilizing medical device 100 to treat a subject, medical device 100 may be introduced into a passageway or an organ of the subject’s body so that distal end 120d of sheath 120 is proximate a target material (e.g., a kidney stone). According to some aspects of the present disclosure, instead of sheath 120 being directly inserted into the subject’s body, another medical device, such as a ureteroscope, may be introduced into the patient’s body and navigated within such that a distal end of the ureteroscope is proximate the target material at the treatment site. Sheath 120 may be extended distally through a working channel of the ureteroscope and may be further extended beyond the distal end of the ureteroscope when the user intends to use medical device 100. After introducing medical device 100, the user may transition end effector 110 of medical device 100 from the collapsed configuration (FIG. 1) to the expanded configuration (FIGS. 2-3) by urging actuators 152, 158 operably coupled to first and second wires 130, 140, respectively, until end effector 110 is extended through the lumen of sheath 120 to be distal to distal end 120d and adopts the predetermined shape of first and second coils 132, 142. After transitioning to the expanded configuration, the user may locate the target material and open the end effector 110 (e.g., by moving first wire 130 away from second wire 140, or vice versa) via actuators 152, 158 until the distance, d, (e.g., the space or opening of end effector 110 between coils 132, 142) permits entry of the target material within volume 114 (e.g., between coils 132, 142). After the target material is within volume 114 via the opening of end effector 110, the user then may close the volume 114 by moving the first wire 130 towards the second wire 140, or vice versa, until the distance, d, between coils 132, 142 prevents the target material from exiting end effector 110.

If the target material is sufficiently sized to be received within sheath 120, the user may retract end effector 110 and the target material via actuators 152, 158. For example, end effector 110 and the target material may be retracted proximally to be within the lumen of sheath 120 (e.g., proximal to distal end 120d of sheath 120). If the target material is too large to be retracted into sheath 120, the user may extend treatment fiber 170 through sheath 120 (e.g., the lumen of sheath 120) to break up the target material within volume 114. For example, a distal end of treatment fiber 170 (e.g., laser fiber) may be extended into volume 114 or until the distal end of treatment fiber 170 is proximate volume 114 and energy applied to break up the size of the target material.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A medical device comprising:
a handle coupled to an end effector, the end effector formed from a first wire and a second wire each forming a coil, the coil of the first wire being proximal to the coil of the second wire;
wherein the first wire and the second wire each comprises a shape memory material, such that the end effector includes a collapsed configuration when constrained within a sheath and an expanded configuration when unconstrained by the sheath;
wherein the first wire and the second wire are movable relative to each other to provide an opening between the coil of the first wire and the coil of the second wire while in the expanded configuration.

2. The medical device of claim 1, wherein the handle includes a first actuator operably coupled to the first wire, the first actuator being configured to move the first wire relative to the second wire.

3. The medical device of claim 1, wherein the handle includes a second actuator operably coupled to the second wire, the second actuator being configured to move the second wire relative to the first wire.

4. The medical device of claim 1, wherein the handle includes a first actuator operably coupled to the first wire and a second actuator operably coupled to the second wire, wherein the first wire and the second wire are movable independent of each other via the first actuator and the second actuator, respectively.

5. The medical device of claim 1, further comprising the sheath, wherein the sheath is coupled to a distal end of the handle and defines a lumen that receives the first wire and the second wire.

6. The medical device of claim 5, wherein the handle includes a port defining a channel in communication with the lumen of the sheath.

7. The medical device of claim 1, wherein the first wire and the second wire are spaced apart at a proximal end of the end effector to permit insertion of a treatment fiber into the end effector in the expanded configuration of the end effector.

8. The medical device of claim 1, wherein the shape memory material comprises nitinol.

9. The medical device of claim 1, wherein a pitch of the coil of the first wire or the coil of the second wire ranges from about 1.5 mm to about 2 mm.

10. The medical device of claim 1, wherein, in the expanded configuration, the end effector includes an ellipsoidal shape.

11. The medical device of claim 1, wherein, in the expanded configuration, a portion of the second wire extends through an interior volume of the coil of the first wire.

12. The medical device of claim 1, wherein, in the expanded configuration, the end effector defines a volume configured to receive a target material, the volume including the opening, wherein a size of the opening is variable by moving the first wire relative to the second wire.

13. A medical device comprising:
a handle including a first actuator and a second actuator;
an end effector formed from a first wire and a second wire each comprising a shape memory material forming a coil, the coil of the first wire being proximal to the coil of the second wire, wherein the first wire is operably coupled to the first actuator and the second wire is operably coupled to the second actuator; and
a sheath coupled to the handle and defining a lumen, the end effector being slidable along the lumen;
wherein the end effector includes a collapsed configuration when constrained within the sheath and an expanded configuration when unconstrained by the sheath; and
wherein the first wire and the second wire are movable relative to each other via the first actuator and the second actuator, respectively, to provide a space between the coil of the first wire and the coil of the second wire while in the expanded configuration.

14. The medical device of claim 13, wherein the handle includes a port defining a channel in communication with the lumen of the sheath.

15. The medical device of claim 13, wherein in the expanded configuration, the end effector defines a volume configured to receive a target material, the volume including an opening defined by a distance between the coil of the first wire and the coil of the second wire, wherein a size of the opening is variable by moving the first wire relative to the second wire.

16. The medical device of claim 13, wherein the coil of the second wire is distal to the coil of the first wire.

17. The medical device of claim 13, wherein each of the first actuator and the second actuator includes a slider.

18. A method of treating a subject, the method comprising:
navigating a distal end of a medical device through a urinary tract of the subject to a treatment site;
extending an end effector of the medical device through a lumen of a sheath of the medical device to exit the lumen, thereby transitioning the end effector from a collapsed configuration to an expanded configuration, wherein the end effector includes a first wire and a second wire each comprising a shape memory material and forming a coil in the expanded configuration, wherein the coil of the first wire is proximal to the coil of the second wire;
moving one of the first wire or the second wire away from the other of the first wire or the second wire;
capturing a target material within a volume of the end effector via an opening between the coil of the first wire and the coil of the second wire; and
moving one of the first wire or the second wire towards the other of the first wire or the second wire to close the opening.

19. The method of claim 18, wherein the medical device comprises a handle coupled to the sheath, the handle including a first actuator operably coupled to the first wire and a second actuator operably coupled to the second wire, the first wire being movable relative to the sheath and independent of movement of the second wire.

20. The method of claim 18, further comprising inserting a treatment fiber into the lumen of the sheath and extending the treatment fiber proximate the end effector before or after capturing the target material within the volume of the end effector.
